(19) United States
(12) Patent Application Publication
(10) Pub. No.: US 2026/0144568 A1
(43) Pub. Date: May 28, 2026

(54) REMOVAL MODULE OF TISSUE REMOVAL APPARATUS

(71) Applicant: LABNPEOPLE CO.,LTD., Gyeonggi-do (KR)

(72) Inventors: Sung Youn CHO, Gyeonggi-do (KR); Duk Su CHOI, Gyeonggi-do (KR)

(21) Appl. No.: 19/121,800

(22) PCT Filed: Oct. 26, 2023
(86) PCT No.: PCT/KR2023/016695

§ 371 Date: Apr. 17, 2025

(30) Foreign Application Priority Data
Oct. 28, 2022 (KR) 10-2022-0141485

(51) Int. Cl. A61B 17/3209; A61B 17/00; A61B 17/32; A61B 17/34

(52) U.S. Cl. CPC A61B 17/3209; A61B 2017/00367; A61B 2017/320028; A61B 2017/3454

(57) ABSTRACT

Proposed is a removal module of a tissue removal apparatus including a bending portion which is connected to a distal end of a catheter, and which is bent with a predetermined curvature by the user's manipulation at the distal end of the catheter; and a tissue removal portion which is exposed on the bending portion along with a shaft that moves forward and backward by user's manipulation when the bending portion is bent. Herein, the tissue removal portion, when exposed to the outside at a distal end of a trocar and then inserted into the inside of the trocar, is inserted into the inside of the trocar while being deformed so as not to be snagged on a distal end side of the trocar.

### TECHNICAL FIELD

The present disclosure relates to a removal module of a tissue removal apparatus configured to easily remove tissue which compresses a nerve or causes diseases by being inserted into a narrow or tubular space within the body.

### BACKGROUND ART

During various surgical procedures, it is often necessary to remove abnormal tissue or bone that has developed in the body. For example, for treatment of diseases such as spinal stenosis, prostatic hyperplasia, and adenomyosis, it is necessary to perform a procedure for removal of not only soft tissue but also hard tissue within the body.

To treat these diseases, various procedural methods such as balloon dilatation, spinal fusion, neuroplasty, and foraminal expansion are used.

For example, balloon dilatation is a procedure to release tissue adhesions and relieve direct pressure on the nerves by placing a balloon in a narrowed nerve root passageway using a catheter and inflating the balloon to widen the nerve root passageway. Balloon dilatation has the advantage of being simple to perform and allowing patients to recover quickly, but balloon dilatation has the limitation of being mainly performed on patients with mild symptoms as balloon dilatation is difficult to fundamentally treat areas with adhesions and show a high recurrence rate.

The spinal fusion can be referred to as a procedural method which fixes movement between two vertebrae to maintain a predetermined distance between the two vertebrae. This procedure usually involves a wide incision. Even when this procedural method is used in a form of a microinvasive procedure, the spinal fusion has the disadvantage of taking longer than other recent microinvasive procedures. In addition, an implant is also inserted to fix the vertebral bodies. Due to that, the vertebral bodies fuse, reducing reduces the patient's mobility. As a result, there are disadvantages of an increase in instability of adjacent segments in the long term and requirement of high costs and long-term care. Accordingly, other problems follow that pose a significant economic and physical burden.

Neuroplasty can be referred to as a procedural method that involves inserting a soft catheter along the spinal canal that extends through the spine and then spraying medication on a pain-causing lesion. This procedure is a microinvasive procedure, so there is an advantage that patients who undergo the procedure recover quickly. However, this procedure is not a fundamental solution to the stenosis-causing structural problem. In addition, this procedure is difficult to accompany physical therapy, so the procedure does not show a significant symptom improvement effect.

In addition, intervertebral foramen expansion can be referred to as a procedural method that secures a nerve passageway of an intervertebral foramen by scraping away biological tissue which has narrowed the intervertebral foramen with a procedural tool equipped with a hard penetration tube. The advantage is that this procedure can be performed while visually confirming the treatment area with an endoscope. However, despite its possibility to remove the tissue on the outside of the intervertebral foramen, the procedure has limitations in scraping off all the tissue inside and outside the three-dimensional intervertebral foramen. Thus, this procedure does not have sufficiently effective function to remove all of the tissue compressing the nerves. Furthermore, this procedure requires the procedural tool and the endoscope to reach the lesion tissue together. Accordingly, tool insertion is required to be performed from more than two directions or the diameter of the tool becomes relatively large. This complicates the procedure, increases the recovery period, and causes increased patient discomfort.

In addition, in performing the procedure mentioned above, there is a trend to perform a minimally invasive surgery method. This is to minimize surgical scars or incision marks by minimizing skin/muscle incisions.

Therefore, the present applicant developed a tissue removal apparatus which is suitable for minimally invasive surgery and enables effective removal of tissues in various locations that cause disease. This tissue removal apparatus is specifically disclosed in Korean Patent No. 10-2300220, which was applied for and registered by the present applicant.

The tissue removal apparatus can be used, for example, to remove lesional tissue directly in the site of intervertebral foraminal stenosis.

The distal end portion of the tissue removal apparatus can be bent toward a lesional tissue inside the body so that the tissue removal apparatus can easily enter the lesional tissue.

In detail, as shown in FIG. 13, the distal end portion of the tissue removal apparatus inserted into the body may include a plurality of bending blocks 20; and a plurality of blades 30 which removes an externally exposed lesional tissue from an internal space formed by the bending blocks 20 when the bending blocks 20 are bent at a predetermined angle.

Here, the plurality of bending blocks 20 may be bent at a predetermined angle, as shown in FIG. 13 when wires W are pulled by a user's manipulation. At this time, the plurality of blades 30 may be exposed to the outside from the internal space formed by the plurality of the bending blocks 20 along with a shaft 40 capable of moving forward/backward by a user's manipulation.

In the state, when a user operates a handle of the tissue removal apparatus to move the shaft 40 forward/backward, the plurality of blades 30 provided at predetermined intervals on the shaft 40 may scrape a lesion tissue.

However, since the distal end portion of the tissue removal apparatus 10 is made of the plurality of bending blocks 20 that are connected and aligned by the wires, there is a risk that excessive tension may cause the wires to break, leaving the bending blocks 20 remaining inside the body.

In addition, the distal end portion and the catheter 50 of the tissue removal apparatus 10 are inserted into the patient's body while passing through a metallic trocar pre-inserted into the patient's lesion site.

In other words, the tissue removal apparatus 10 is configured to safely and accurately reach the lesion site by being guided by a hollowed trocar pre-inserted into the patient's body.

At this time, the distal end portion of the tissue removal apparatus 10, while extending in an exposed state beyond the end of the trocar inserted into the body, may be bent toward the lesional tissue, and then remove the lesion tissue.

However, when the lesion tissue is removed, the distal end portion and the catheter 50 of the tissue removal apparatus 10, as inserted into the trocar, are required to be pulled out from the internal space of the trocar. During this process, there is a problem in that the plurality of blades 30 come into contact with and get snagged on the end of the trocar inserted into the body.

To elaborate, the distal end portion of the tissue removal apparatus 10 is bent toward the to-be-removed tissue while passing through the trocar. Thus, when the user pulls the handle of the tissue removal apparatus 10 to pull out the catheter 50 and the plurality of bending blocks 20 from the inside of the trocar, the problem is that the plurality of blades 30 get snagged on the end of the trocar and cannot be inserted into the internal space of the trocar due to the angle of the bent bending blocks 20 and the position of the blades 30.

In particular, the blades 30 are configured to be multiple, as provided at predetermined intervals along the longitudinal direction of the shaft 40. Therefore, even when one blade 30 is immediately inserted into the internal space of the trocar without being snagged on the end of the trocar, there is a problem in that another blade 30 comes into contact with and gets snagged on the end of the trocar.

Therefore, the user is required to perform the cumbersome action of repeatedly moving the handle of the tissue removal apparatus forward/backward until the plurality of blades 30, exposed beyond the end of the trocar and placed inside the patient's body, are inserted into the internal space of the trocar.

In this process, since the blades 30 repeatedly come into contact with the end of the trocar, there is a problem that the blades 30 are damaged and deformed. More seriously, there is a problem that the blades 30 detach from the shaft 40 and remain inside the patient's body.

Therefore, the present applicant proposed the present disclosure to solve the problems. Related conventional art literature includes Korean Patent NO. 10-2300220, titled “Tissue Removal Apparatus”.

### DISCLOSURE

### Technical Problem

To solve the problems, the present disclosure is to provide a removal module of a tissue removal apparatus which allows a distal end portion of the tissue removal apparatus to be easily inserted into an internal space of a trocar without interference of an end of the trocar, the distal end portion of the tissue removal apparatus being bent at a predetermined angle inside the patient's body.

To solve the problems, the present disclosure is also to provide a removal module of a tissue removal apparatus which is configured to allow a user to quickly address a snagging phenomenon of a blade at the end of the trocar even when the blades of the tissue removal apparatus come into contact with and are snagged on the end of the trocar.

To solve the problems, the present disclosure is also to provide a removal module of a tissue removal apparatus which prevents surgical tool residues from being left inside the patient's body during a process of removing lesion tissue.

### Technical Solution

The present disclosure may include a bending portion, which is connected to a distal end of a catheter, and which is bent with a predetermined curvature by the user's manipulation at the distal end of the catheter; and a tissue removal portion which is exposed on the bending portion along with a shaft that moves forward and backward by user's manipulation when the bending portion is bent. Herein, the tissue removal portion, when exposed to the outside at a distal end of a trocar and then inserted into the inside of the trocar, may be inserted into the inside of the trocar while being deformed so as not to be snagged on a distal end side of the trocar.

In addition, the bending portion may include an insertion member inserted into a hole formed at the distal end of the catheter; and a bending member, which is integrally connected to the insertion member and is bent to a predetermined curvature.

In addition, the bending portion may be formed along the longitudinal direction of the bending member and may include a seating groove on which the shaft passing through the distal end of the catheter is seated.

In addition, the tissue removal portion may include a receiving member, while accommodating the shaft, which is connected to a portion of a circumferential surface of the shaft; a blade, which is connected to the receiving member and which is in contact with the tissue to be removed; and a snagging prevention member, which is provided at a longitudinal end of the receiving member and which is pre-inserted into the inside of the trocar prior to the blade.

In addition, an end of the snagging prevention member may have a downwardly inclined shape.

In addition, an end of the snagging prevention member may come in contact with a compressing member which protrudes toward the inside of the trocar on a distal end surface of the trocar or at the distal end side of the trocar.

In addition, it is possible for the blade to be inserted into the inside of the trocar through an incision groove formed by an incision at the distal end side of the trocar.

### Advantageous Effects

A removal module of a tissue removal apparatus according to an embodiment of the present disclosure enables a bending member bent at a predetermined angle inside the patient's body and a shaft and tissue removal portion exposed to the outside from the bending member to be easily inserted into an internal space of the trocar through a hole formed at a distal end of the trocar.

In addition, the removal module of the tissue removal apparatus according to another embodiment of the present disclosure allows a user to quickly address a snagging phenomenon of a blade at the distal end portion of the trocar, even when the blade comes into contact with and is snagged on the distal end surface of the trocar.

In addition, the removal module of the tissue removal apparatus according to another embodiment of the present disclosure allows for preventing the tissue-excising blade or the bending member from being damaged during the process of removing a lesion-causing tissue, thereby preventing a procedural tool from remaining inside the patient's body.

### DESCRIPTION OF DRAWINGS

FIG. 1 shows a perspective view showing a removal module according to an embodiment of the present disclosure provided at a distal end portion of a tissue removal apparatus;

FIG. 2 shows a perspective view of the removal module according to another embodiment of the present disclosure;

FIG. 3 shows a side view of the removal module according to a further embodiment of the present disclosure;

FIG. 4 shows a perspective view of a bending portion according to a yet further embodiment of the present disclosure;

FIG. 5 shows a side view of the bending portion according to a still yet further embodiment of the present disclosure;

FIG. 6 shows a perspective view of the bending portion according to a still yet further embodiment of the present disclosure viewed from the rear;

FIG. 7 shows a perspective view of a tissue removal portion according to a still yet further embodiment of the present disclosure;

FIG. 8 shows a side view of the tissue removal portion according to a still yet further embodiment of the present disclosure;

FIG. 9 shows a perspective view of the tissue removal portion according to a still yet further embodiment of the present disclosure viewed from the rear;

FIG. 10 shows a side view showing a state of the bending portion shown in FIG. 5, which is bent to a predetermined curvature by the user's manipulation;

FIG. 11 shows a perspective view of a trocar;

FIG. 12 shows a perspective view showing a distal end of the trocar; and

FIG. 13 shows a side view showing the configuration of a conventional removal module.

### MODE FOR DISCLOSURE

Advantages and features of the present disclosure and methods for achieving them will become clear by referring to the embodiments described in detail below along with the accompanying drawings.

However, the present disclosure is not limited to the embodiments disclosed below, but is implemented in various different forms. These embodiments are merely provided to ensure that the disclosure of the present disclosure is complete and to fully inform those skilled in the art of the scope of the invention. The present disclosure is defined only by the scope of the claims.

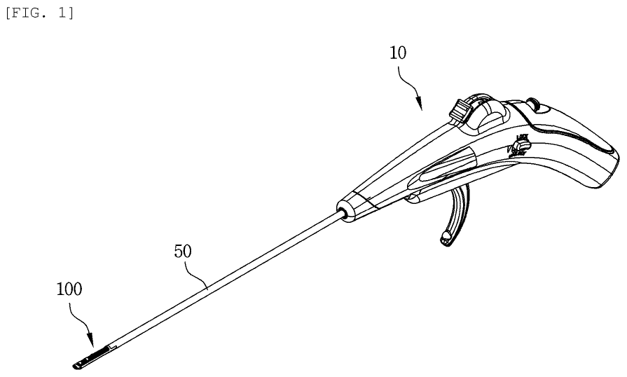

Hereinafter, with reference to FIGS. 1 to 13, a removal module of a tissue removal apparatus according to an embodiment of the present disclosure will be described in detail. In describing the present disclosure, detailed descriptions of related well-known functions or configurations are omitted to make the gist of the invention unambiguous.

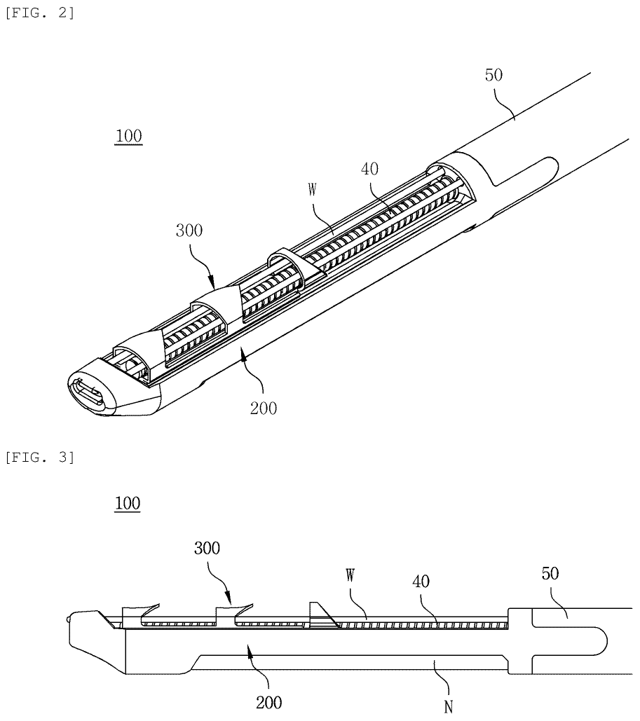

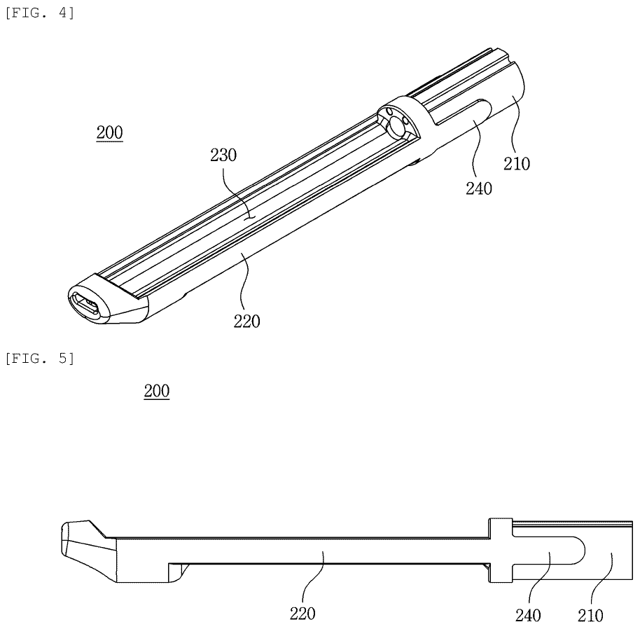

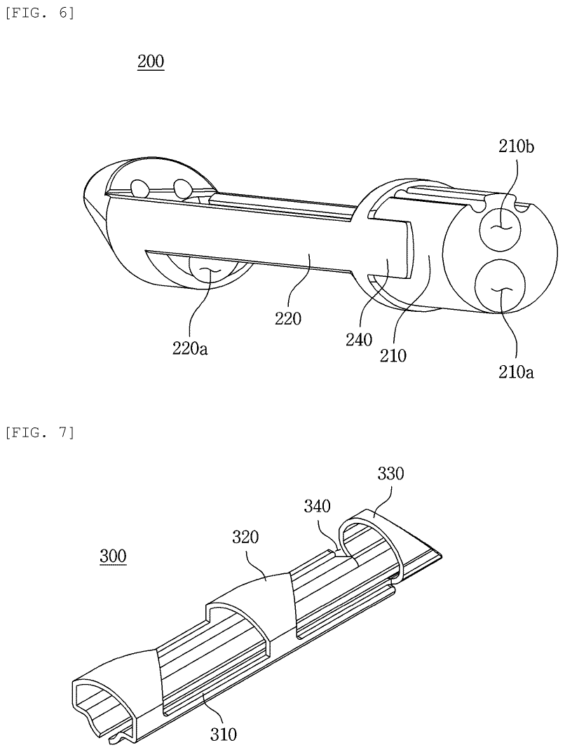

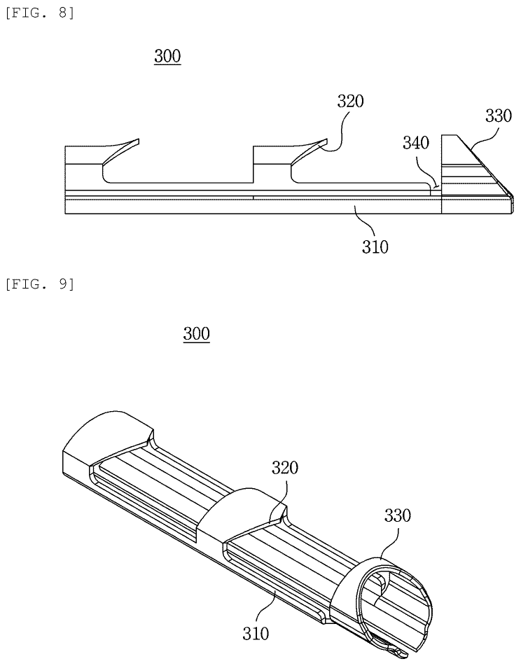

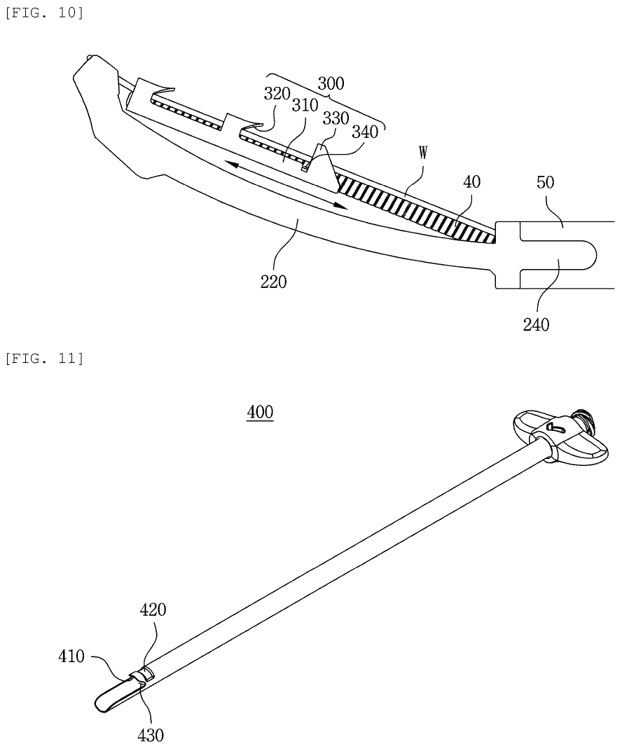

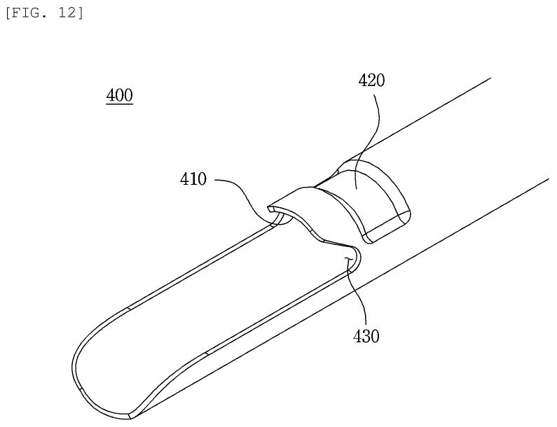

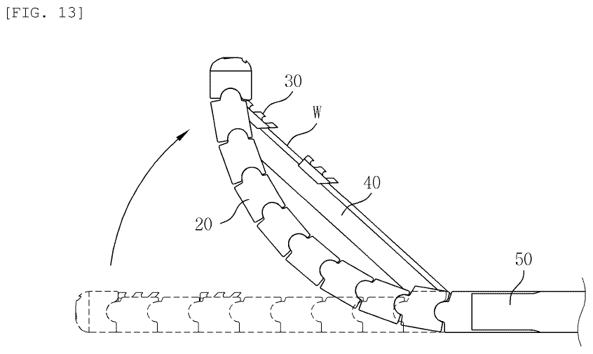

FIG. 1 shows a perspective view showing a removal module according to an embodiment of the present disclosure provided at a distal end portion of a tissue removal apparatus. FIG. 2 shows a perspective view of the removal module according to another embodiment of the present disclosure. FIG. 3 shows a side view of the removal module according to a further embodiment of the present disclosure. FIG. 4 shows perspective view of a bending portion according to a yet further embodiment of the present disclosure. FIG. 5 shows a side view of the bending portion according to a still yet further embodiment of the present disclosure. FIG. 6 shows a perspective view of the bending portion according to a still yet further embodiment of the present disclosure viewed from the rear. FIG. 7 shows a perspective view of a tissue removal portion according to a still yet further embodiment of the present disclosure. FIG. 8 shows a side view of the tissue removal portion according to a still yet further embodiment of the present disclosure. FIG. 9 shows perspective view of the tissue removal portion according to a still yet further embodiment of the present disclosure viewed from the rear. FIG. 10 shows a side view showing a state of the bending portion shown in FIG. 5, which is bent to a predetermined curvature by the user's manipulation. FIG. 11 shows a perspective view of a trocar. FIG. 12 shows a perspective view showing a distal end of the trocar. FIG. 13 shows a side view showing the configuration of a conventional removal module.

As shown in FIG. 1, a removal module 100 according to an embodiment of the present disclosure may be provided at a distal end portion of a tissue removal apparatus 10. That is, the removal module 100 is provided at a distal end of a catheter 50 that constitutes the tissue removal apparatus 10, and may be inserted into the body to remove lesion-causing tissue.

In FIGS. 2 and 3, the removal module 100 is shown in more detail.

The removal module 100, as shown in FIGS. 2 and 3, may include a bending portion 200, which is connected to a distal end of a catheter 50, and which is bent with a predetermined curvature by the user's manipulation at the distal end of the catheter 50; and a tissue removal portion 300 which is mounted on a shaft 40 disposed on a seating groove of the bending portion 200 and which is exposed on the bending portion 200 along with the shaft 40 when the bending portion 200 is bent.

The bending portion 200, as shown in FIGS. 4 to 6, may include an insertion member 210 inserted into a hole formed at the distal end of the catheter 50; a bending member 220, which is integrally connected to the insertion member 210 and is bent to a predetermined curvature; and a seating groove 230 on which the shaft 40 passing through the distal end of the catheter 50 is seated.

The insertion member 210 may have an overall cylindrical shape.

In addition, as shown in FIG. 6, on the upper surface of the insertion member 210, a pair of “U”-shaped grooves may be formed, through which wires W that exert tension to bend the bending member 200 pass.

In addition, in the insertion member 210, a first hole 210a which the tube N (see FIG. 3) is inserted into and passes through, and a second hole 210b which the shaft 40 is inserted into and passes through may be formed, respectively. For reference, the tube N may be used for irrigation or drug delivery.

Additionally, a rotation prevention member 240 may be formed on a circumferential surface of the insertion member 210.

When the insertion member 210 is inserted into the hole formed at the distal end of the catheter 50, the rotation prevention members 240 may be inserted into a pair of movement prevention grooves formed at the distal end of the catheter 50 to prevent the insertion member 210 from rotating.

The bending member 220 has a predetermined length and, as described above, is integrally connected with the insertion member 210.

In the distal end portion of the bending member 220, a hole 220a into which the tube N that has passed through the second hole 210b of the insertion member 210 may be inserted, may be formed.

In addition, as shown in FIGS. 2 and 3, a pair of holes may be formed in the distal end portion of the bending member 220. Herein, the wires W that have passed through the insertion member 210 may be inserted into and pass through the pair of holes. That is, the wires W sequentially may pass through the pair of holes formed at the distal end of the bending member 220, and then may pass through the U-shaped grooves formed on the upper surface of the insertion member 210, and finally may be moved inside a handle of the tissue removal apparatus 10.

Therefore, when a user manipulates the handle of the tissue removal apparatus 10 and pulls the wires W, the bending member 220 may be bent to a predetermined curvature.

The seating groove 230 formed in the bending member 220 may be referred to as a space where a tissue removal portion 300, which will be described later, and a shaft 40 linearly reciprocating the tissue removal portion 300 are disposed.

The bending portion 200, as configured above, serves to guide the tissue removal portion 300 to a pain-causing tissue at a lesion site in the body. In particular, the bending portion 200 enables the forward and backward movement of the tissue removal portion 300 to be performed more stably in a tubular structure of the body or in narrow areas within the body.

In other words, when the bending member 220 of the bending portion 200 is bent to a predetermined curvature within the body, the bent bending member 220 serves as a support point. Due to that, not only does it increase adhesion between the tissue removal portion 300, which will be described later, and the tissue to be removed, but the tissue removal portion 300 moves forward and backward stably.

In addition, the bending portion 200 is preferably made of a material that may be bent to have a predetermined curvature and then restored to a straight shape. That means that the bending portion 200 may be made of a plastic or metal material with excellent elastic recovery force or flexibility.

The tissue removal portion 300, when exposed to the outside at the distal end of the trocar 400 and then inserted into the hole formed at the distal end of the trocar 400, may be deformed by being compressed against a distal end side of the trocar 400 so as not to be snagged on the distal end side of the trocar 400.

The tissue removal portion 300 as described above, as shown in FIGS. 7 to 9, may include a receiving member 310, while accommodating a portion of the shaft 40 in the longitudinal direction, disposed on the seating groove 230 formed in the bending member 220 and joined to a portion of the circumferential surface of the shaft 40; a blade 320 exposed to the upper part of the seating groove 230 while connected to the receiving member 310, and in contact with the tissue to be removed; and a snagging prevention member 330 that is formed by being integrally connected with the receiving member 310 at the longitudinal end of the receiving member 320 and is inserted into the internal space of the trocar 400 prior to the blade 320.

The receiving member 310 may be connected to a portion of the circumferential surface f the shaft 40 using a known joining method. For example, the receiving member 310 may be connected to the shaft 40 through laser welding, or may be connected to the shaft 40 through a known compressing method.

For reference, the shaft 40 is preferably made of a material that may be bent to have a predetermined curvature and then restored to a straight shape. That means that the shaft 40 may be made of a plastic or metal material with excellent elastic recovery force or flexibility.

Additionally, the receiving member 310 may be provided as a pair and may have a bent shape to have a curvature confirming to a portion of the circumferential surface of the shaft 40.

The blade 320 is provided in a form that connects a pair of receiving members 310 to each other, and is arranged to be in non-contact with the shaft 40 joined to the pair of receiving members 310.

At least one blade 320 may be provided on the pair of receiving members 310 in numbers. According to an embodiment of the present disclosure, a drawing shows that a pair of blades 320 are provided on the pair of receiving members 310 at a predetermined distance from each other.

The blade 320 having the configuration is a component that directly comes into contact with the tissue to be removed, and one end thereof is sharpened to scrape or excise the tissue.

Additionally, as shown in FIG. 8, one end of the blade 320 may be bent upward at a predetermined angle to easily scrape or excise tissue.

Therefore, when the shaft 40 is linearly reciprocated by the user's manipulation, the pair of receiving members 310 joined to the shaft 40 are also linearly reciprocated, so the blade 320 may scrape or excise lesion-causing tissue.

When snagging prevention member 330, when the tissue removal portion 300 exposed to the outside at the distal end of the trocar 400 (see FIG. 12) is inserted into the internal space of the trocar 400, serves to prevent the blade 320 from being snagged on the distal end surface 410 of the trocar 400.

To insert the bending portion 200 and tissue removal portion 300 exposed to the outside at the distal end of the trocar 400 into the internal space of the trocar 400, when the user pulls the catheter 50 using the handle of the tissue removal apparatus 10, the bending portion 200 and the tissue removal portion 300 may be inserted into the hole formed at the distal end of the trocar 400.

When the bending member 220 of the bending portion 200 is not bent to a predetermined curvature and maintains a straight shape, the bending portion 200 may be easily inserted into the internal space of the trocar 400.

However, as shown in FIG. 10, when the bending member 220 is bent at a predetermined curvature, and because of this, the shaft 40 and the tissue removal portion 300 are exposed to the outside in the seating groove 230 of the bending member 220, it is difficult to insert the shaft 40 and the tissue removal portion 300 into the internal space of the trocar 400. This is because, in the state shown in FIG. 10, the blade 320 of the tissue removal portion 300 comes into contact with and is snagged on the distal end surface 410 of the trocar 400.

The handle of the tissue removal apparatus 10 may be manipulated so that the bending member 220 has a straight shape. However, there are problems in that, in tubular spaces such as blood vessels in the body or narrow spaces, it is not only difficult to return the bending member 220 to its original state once bent, but also normal tissues or nerves in the body be contacted and injured during the process of deforming the bending member 220 to its original state.

Therefore, it is preferable to pre-insert the bending member 220 bent to a predetermined curvature and the tissue removal portion 300 exposed to the outside on the bending member 220 into the internal space of the trocar 400 through a hole formed at the distal end of the trocar 400. That is, it is preferable to insert the bending member 220 and the tissue removal portion 300 into the hole formed at the distal end of the trocar 400, as in the state shown in FIG. 10.

As described above, the snagging prevention member 330 may be inserted into the hole formed at the distal end of the trocar 400 prior to the blade 320.

In this process, even when one end of the snagging prevention member 330 comes in contact with the distal end surface 410 of the trocar 400, one end of the snagging prevention member 330 is inclined downward so that the snagging prevention member 330 may be easily inserted into the internal space of the trocar 410.

Therefore, even when one end of the inclined snagging prevention member 330 comes into contact with the distal end surface 410 of the trocar 400, the snagging prevention member 330 may be easily inserted into the internal space of the trocar 400.

At this time, when the end of the inclined snagging prevention member 330 is compressed by an external force, the receiving member 310 integrally connected with the snagging prevention member 330 is also compressed and deformed. Thus, the blade 320 integrally connected with the receiving member 310 is deformed, thereby lowering the overall height.

Then, the blade 320 is deformed to a height where the blade 320 is not snagged on the distal end surface 410 of the trocar 400, so the blade 320 may be easily inserted into the trocar 400 without interference of the distal end surface 410.

Herein, the snagging prevention member 330 and the blade 320 preferably have a deformable shape when an external force is applied.

For example, as shown in FIG. 9, the snagging prevention member 330 and the blade 320 may be manufactured in cooperation with the receiving member 310 to form a space through which the shaft 40 and the wires W may pass.

Additionally, the end of the inclined snagging prevention member 330 may come in contact with the distal end surface 410 of the trocar 400 or the compressing member 420 shown in FIG. 12.

Herein, the compressing member 420 may be formed to protrude from the outer surface of the trocar 400 toward the internal space. Accordingly, one end of the inclined snagging prevention member 330 may come into contact with the portion of the compressing member 420 disposed in the internal space of the trocar 400 during the process of inserting the snagging prevention member 330 into the hole formed at the distal end of the trocar 400.

When the snagging prevention member 330 is compressed by the compressing member 420, as described above, the receiving member 310 integrally connected with the snagging prevention member 330 is also compressed and deformed. In addition, the blade 320 integrally connected with the receiving member 310 may be deformed into a compressed form.

Then, the blade 320 is deformed to a height where the blade 320 is not snagged on the distal end surface 410 of the trocar 400, so the blade 320 may be easily inserted into the trocar 400 without interference of the distal end surface 410.

Meanwhile, even when the snagging prevention member 330 is easily inserted through the hole formed at the distal end of the trocar 400, the end of the blade 320 may come into contact with and become snagged on the distal end surface 410 of the trocar 410.

At this time, even when the blade 320 comes into contact with and is snagged on the distal end surface 410 of the trocar 400, a user may insert the blade 320 into the internal space of the trocar 430 through an incision groove 430 formed by an incision at the distal end side of the trocar 400.

That is, when the blade 320 is snagged on the distal end surface 410 of the trocar 400, an operator may rotate the catheter 50 inserted into the trocar 400 using the handle of the tissue removal apparatus 10. Then, the tissue removal portion 30 is also rotated in conjunction with the rotation of the catheter 50. Because of that, the blade 320 snagged on the distal end surface 410 of the trocar 400 may be rotated and inserted into the internal space of the trocar 400 through the incision groove 430 shown in FIG. 13.

The removal module 100 of the tissue removal apparatus according to an embodiment of the present disclosure enables a bending member 220 bent at a predetermined angle inside the patient's body, and a shaft 40 and tissue removal portion 300 exposed to the outside from the bending member 220 to be easily inserted into an internal space of the trocar 400 through a hole formed at a distal end of the trocar 400.

In addition, the removal module 100 of the tissue removal apparatus according to an embodiment of the present disclosure allows a user to quickly address the snagging phenomenon of the blade 320 at the distal end of the trocar 400, even when the blade 320 comes into contact with and is snagged on the distal end of the trocar 400.

In addition, the removal module 100 of the tissue removal apparatus according to another embodiment of the present disclosure allows for preventing the tissue-excising blade 320 or the bending member 220 from being damaged during the process of removing a lesion-causing tissue, thereby preventing a procedure tool from remaining inside the patient's body.

Although specific embodiments according to the present disclosure have been described so far, it is obvious that various modifications are possible without departing from the scope of the present disclosure.

For example, the snagging prevention member 330 may be made of a different material from the receiving member 310 and may be provided at the longitudinal end of the receiving member 310. That is, the snagging prevention member 300 may, of course, be manufactured separately and connected to the end of the receiving member 310.

Therefore, the scope of the present disclosure should not be limited to the described embodiments, but should be determined by the scope of the patent claims described later as well as equivalents to the claims of this patent.

### Industrial Applicability

The present disclosure may be applied and sold in the medical industry.

1. A removal module of a tissue removal apparatus, the removal module comprising:
a bending portion, which is connected to a distal end of a catheter, and which is bent with a predetermined curvature by the user's manipulation at the distal end of the catheter; and
a tissue removal portion which is exposed on the bending portion along with a shaft that moves forward and backward by user's manipulation when the bending portion is bent,
wherein the tissue removal portion, when exposed to the outside at a distal end of a trocar and then inserted into the inside of the trocar, is inserted into the inside of the trocar while being deformed so as not to be snagged on a distal end side of the trocar.

2. The removal module of claim 1, wherein the bending portion comprises:
an insertion member inserted into a hole formed at the distal end of the catheter; and
a bending member, which is integrally connected to the insertion member and is bent to a predetermined curvature.

3. The removal module of claim 2, wherein the bending portion is formed along the longitudinal direction of the bending member and comprises a seating groove on which the shaft passing through the distal end of the catheter is seated.

4. The removal module of claim 1, wherein the tissue removal portion comprises:
a receiving member, while accommodating the shaft, which is joined to a portion of a circumferential surface of the shaft;
a blade, which is connected to the receiving member and which is in contact with the tissue to be removed; and
a snagging prevention member, which is provided at a longitudinal end of the receiving member and which is pre-inserted into the inside of the trocar prior to the blade.

5. The removal module of claim 4, wherein an end of the snagging prevention member has a downwardly inclined shape.

6. The removal module of claim 4, wherein an end of the snagging prevention member comes in contact with a compressing member which protrudes toward the inside of the trocar on a distal end surface of the trocar or at the distal end side of the trocar.

7. The removal module of claim 4, wherein it is possible for the blade to be inserted into the inside of the trocar through an incision groove formed by an incision at the distal end side of the trocar.
